(19) United States
(12) Patent Application Publication
(10) Pub. No.: US 2026/0159547 A1
(43) Pub. Date: Jun. 11, 2026

(54) SHORT-CHAIN ANTIBACTERIAL PEPTIDE VKLR, PREPARATION METHOD AND APPLICATIONS THEREOF

(71) Applicant: Huazhong Agricultural University, Wuhan City (CN)

(72) Inventors: Haihong HAO, Wuhan City (CN); Yakun DING, Wuhan City (CN)

(21) Appl. No.: 19/411,578

(22) Filed: Dec. 8, 2025

(30) Foreign Application Priority Data
Dec. 11, 2024 (CN) 202411818666.8

(51) Int. Cl. C07K 7/08

(52) U.S. Cl. CPC C07K 7/08

(57) ABSTRACT

A short-chain antibacterial peptide VKLR, a preparation method and applications thereof are provided. The short-chain antibacterial peptide VKLR has an amino acid sequence as shown in SEQ ID No. 1. VKLR is a short-chain antibacterial peptide composed of natural amino acids and possesses an antibacterial activity. The VKLR shows no cytotoxicity in in vitro cytotoxicity experiments. The antibacterial peptide has a mature preparation process, and may be produced by fermentation using an engineering bacterium expression system.

### CROSS-REFERENCE TO RELATED APPLICATIONS

This application claims priority to Chinese Patent Application No. 202411818666.8, filed on Dec. 11, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Dec. 4, 2025
Byte size: 1,838

### TECHNICAL FIELD

The present disclosure relates to the technical field of agricultural, animal husbandry and veterinary applications, and particularly relates to a short-chain antibacterial peptide VKLR, a preparation method and applications thereof.

### BACKGROUND

As one of the most important medical discoveries in the 20th century, antibiotics have saved countless lives and made significant contributions to the prevention and treatment of human infectious diseases. Approximately 70% of the antibacterial agents used in human medicine today are obtained during the golden age of antibiotic discovery. Most of these drugs are isolated from actinomycetes and fungi. However, after the 1970s, it has become very difficult to obtain valuable antibiotics from microbial metabolites under laboratory conditions using the method of pure microbial culture. Current research and development of antibacterial agents mostly focus on the modification of known compounds or their optimized combinations. During this period when discovering new antibiotics is becoming increasingly difficult, bacterial resistance to antibiotics is increasing year by year. Antibiotic resistance enables bacteria to tolerate antibiotics at clinical treatment concentrations, causing antibiotics to lose their therapeutic efficacy. It is predicted that by 2050, the number of human deaths caused by multidrug resistance will increase to 10 million, surpassing the number of deaths from cancer and becoming one of the leading causes of human death globally. Therefore, it is extremely urgent to control the development of drug resistance.

As a naturally sourced biological material, antibacterial peptides have become popular candidates for antibacterial agents, especially as substitutes for veterinary antibiotics, in recent years due to their unique mechanism of disrupting pathogenic biofilms. However, naturally occurring antibacterial peptides have a series of problems such as weak biological activity and high toxicity. The problems severely limit and hinder their translational application. Therefore, how to obtain an antibacterial peptide with high biological activity and low toxicity is of great significance.

### SUMMARY

The purpose of the present disclosure is to provide a short-chain antibacterial peptide VKLR, its preparation method and application, to solve the problems existing in the above-mentioned prior art. The present disclosure provides an antibacterial peptide VKLR with high antibacterial activity, which contains only 13 amino acids, has low synthesis cost, and possesses excellent broad-spectrum antibacterial activity. The antibacterial peptide not only has significant bacteriostatic effects, but also shows no cytotoxicity in in vitro cytotoxicity experiments, indicating extremely high safety.

To achieve the above purpose, the present disclosure provides the following schemes.

Technical scheme 1: A highly active short-chain antibacterial peptide VKLR, whose amino acid sequence is as shown in SEQ ID NO. 1, with the amino acid sequence as follows: LVKLRVKLRVKLR.

The present disclosure uses the following technical scheme for screening and obtaining: 1) collecting all antibacterial peptide sequences with antibacterial function from the antibacterial peptide database (https://aps.unmc.edu/AP/); 2) calculating the amino acid composition, secondary structure, sequence length, positive charge count, hydrophobicity, and adjacent amino acid abundance for all sequences; 3) based on the above parameters, rationally designing and synthesizing a batch of antibacterial peptides; and 4) screening for one antibacterial peptide VKLR with excellent antibacterial activity and the lowest cytotoxicity based on in vitro antibacterial activity, cytotoxicity, and hemolytic activity.

Technical scheme 2: An application of the highly active short-chain antibacterial peptide VKLR in any one of the following, including:
(1) preparing a broad-spectrum antibacterial agent;
(2) preparing a preservative;
(3) preparing an antibacterial drug or an antibacterial composition; and
(4) preparing a medical dressing.

In an embodiment, the antibacterial activity includes activity against Gram-negative bacteria and/or Gram-positive bacteria.

In an embodiment, the antibacterial activity includes activity against Escherichia coli, Pseudomonas aeruginosa, Staphylococcus aureus and/or Staphylococcus epidermidis.

Technical scheme 3: A broad-spectrum antibacterial agent. The broad-spectrum antibacterial agent contains the highly active short-chain antibacterial peptide VKLR.

Technical scheme 4: A preservative. The preservative contains the highly active short-chain antibacterial peptide VKLR.

Technical scheme 5: An antibacterial drug or antibacterial composition. The antibacterial drug or antibacterial composition contains the highly active short-chain antibacterial peptide VKLR.

Technical scheme 6: A medical dressing. The medical dressing contains the highly active short-chain antibacterial peptide VKLR.

Technical scheme 7: An application of the highly active short-chain antibacterial peptide VKLR in preparing medical imaging agents, daily chemical detergents and/or medical devices having antibacterial activity.

The present disclosure discloses the following technical effects.

Based on an antibacterial peptide database, the present disclosure designs and synthesizes a highly active short-chain antibacterial peptide VKLR through database filtering technology and computer-aided design, and by optimizing the amino acid arrangement, maximally reduces its hemolytic activity while maintaining high antibacterial activity. The results of the embodiments show that the antibacterial peptide of the present disclosure not only has significant bacteriostatic effects, but also shows no cytotoxicity in in vitro cytotoxicity experiments, indicating extremely high safety. The antibacterial peptide of the present disclosure has a mature preparation process, convenient access channels, and may be produced by fermentation using an engineering bacterium expression system.

### BRIEF DESCRIPTION OF THE DRAWINGS

In order to more clearly illustrate the technical schemes in the embodiments of the present disclosure or the prior art, the drawings required for use in the embodiments will be briefly introduced below. Apparently, the drawings in the following description are only some embodiments of the present disclosure. For those of ordinary skill in the art, other drawings may also be obtained from these drawings without creative efforts.

FIG. 1 shows the mass spectrum of the antibacterial peptide VKLR.

FIG. 2 shows the bactericidal kinetic curve of the antibacterial peptide VKLR against Pseudomonas aeruginosa.

FIG. 3 shows the bactericidal kinetic curve of the antibacterial peptide VKLR against Staphylococcus aureus.

FIG. 4 shows the bar graph of the effect of the antibacterial peptide VKLR on bacterial outer membrane permeability.

FIG. 5 shows the bar graph of the affinity of the antibacterial peptide VKLR for Lipopolysaccharide (LPS)/Lipoteichoic acid (LTA).

FIG. 6 shows the result of the inner membrane depolarization of Pseudomonas aeruginosa by the antibacterial peptide VKLR.

FIG. 7 shows the result of the inner membrane depolarization of Staphylococcus aureus by the antibacterial peptide VKLR.

FIG. 8 shows the result of the hemolysis rate of red blood cells by the antibacterial peptide VKLR.

### DETAILED DESCRIPTION OF THE EMBODIMENTS

Various exemplary embodiments of the present disclosure are described in detail below. This detailed description should not be construed as limiting the disclosure, but should be understood as a more detailed description of certain aspects, characteristics, and embodiments of the disclosure.

It should be understood that the terms used in the present disclosure are only for describing specific embodiments and are not intended to limit the disclosure. Furthermore, for numerical ranges in the present disclosure, it should be understood that each intermediate value between the upper and lower limits of the range is specifically disclosed. Every intermediate value within any stated value or stated range, and every smaller range between any other stated value or intermediate value within the stated range, is also included in the present disclosure. The upper and lower limits of these smaller ranges may be independently included or excluded from the range.

Unless otherwise stated, all technical and scientific terms used herein have the same meaning as commonly understood by one of ordinary skill in the art to which this disclosure belongs. Although the present disclosure describes only optional methods and materials, any methods and materials similar or equivalent to those described herein may also be used in the practice or testing of the present disclosure. All documents mentioned in this specification are incorporated by reference to disclose and describe methods and/or materials related to the respective documents. In the event of conflict with any incorporated document, the content of this specification shall prevail.

Various modifications and changes to the specific embodiments described in the specification of the present disclosure may be made without departing from the scope or spirit of the disclosure, which will be apparent to those skilled in the art. Other embodiments obtained from the description of the present disclosure will be apparent to those skilled in the art. The specification and examples of the present disclosure are exemplary only.

Regarding the terms “comprising”, “including”, “having”, “containing”, etc., used herein, they are all open-ended terms, meaning including but not limited to.

### Embodiment 1 Design of the Antibacterial Peptide

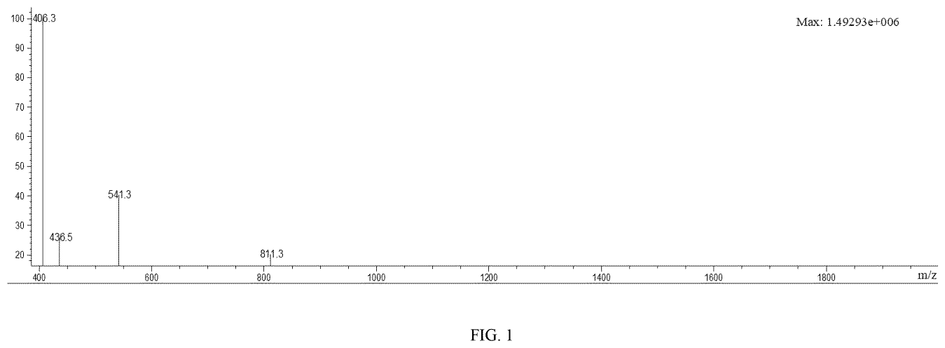

(1) All antibacterial peptide sequences with antibacterial function are collected from the antibacterial peptide database (https://aps.unmc.edu/AP/). (2) The amino acid composition, secondary structure, sequence length, positive charge count, hydrophobicity, and adjacent amino acid abundance of all sequences are calculated. (3) Based on the above parameters, a batch of antibacterial peptides is rationally designed and synthesized. (4) One antibacterial peptide VKLR with excellent antibacterial activity and the lowest cytotoxicity is screened based on in vitro antibacterial activity, cytotoxicity, and hemolytic activity. FIG. 1 shows the mass spectrum of the antibacterial peptide VKLR. The sequence and physicochemical parameters of the antibacterial peptide VKLR are detailed in Table 1.

TABLE 1

| | | Theoretical | Actual | | | |
|---|---|---|---|---|---|---|
| | | molecular | molecular | Charge | | Hydrophobic |
| Peptide | Sequence | weight | weight | number | Hydrophobicity | moment |
| VKLR | LVKLRVKLRVKLR | 1621.14 | 1621.11 | +6 | 0.343 | 0.322 |
| | (SEQ ID NO. 1) | | | | | |

### Embodiment 2 Synthesis of the Antibacterial Peptide VKLR by Solid-Phase Chemical Synthesis

The synthesis method is as follows.

The polypeptides of the present disclosure are all prepared by solid-phase synthesis.

(1) The preparation of the antibacterial peptide is carried out from the C-terminus to the N-terminus one by one using a peptide synthesizer. First, Fmoc-X (X is the first amino acid at the C-terminus of each antibacterial peptide) is loaded onto Wang resin, and then the Fmoc group is removed to obtain X-Wang resin. Then Fmoc-Y-Trt-OH (9-fluorenylmethoxycarbonyl-trimethyl-Y, Y is the second amino acid at the C-terminus of each antibacterial peptide) is added. According to this procedure, synthesis is sequentially performed from the C-terminus to the N-terminus until completion, and the side-chain protected peptide resin with the Fmoc group removed is obtained.

(2) To the peptide resin obtained above, a cleavage reagent is added, and the reaction is carried out at 20 degrees Celsius protected from light for 2 hours, followed by filtration. The precipitate is washed with trifluoroacetic acid (TFA). The wash solution is combined with the aforementioned filtrate, concentrated with a rotary evaporator, then about 10 times the volume of pre-cooled anhydrous ether is added, and precipitation is carried out at −20 degrees Celsius for 3 hours, yielding a white powder, which is centrifuged at 2500 times gravity for 10 minutes. The precipitate is collected, washed again with anhydrous ether, and dried under vacuum to obtain the polypeptide. The cleavage reagent is a mixture of TFA, water, and triisopropylsilane (TIS) in a mass ratio of 95:2.5:2.5.

(3) The column is equilibrated with 0.2 molar sodium sulfate (adjusted to pH 7.5 with phosphoric acid) for 30 minutes. The polypeptide is dissolved in 90% acetonitrile aqueous solution, filtered, and purified using a C18 reverse-phase normal-pressure column with gradient elution (the eluent is a mixture of methanol and sodium sulfate aqueous solution in a volume ratio ranging from 30:70 to 70:30). The flow rate is 1 milliliter per minute. The detection wavelength is 220 nanometers. The main peak is collected, and lyophilized. Further purification is performed using a reverse-phase C18 column, where eluent A is a 0.1% TFA/water solution and eluent B is a 0.1% TFA/acetonitrile solution. The elution concentration is from 25% B to 40% B. The elution time is 12 minutes, the flow rate is 1 milliliter per minute, and the main peak is collected as above and lyophilized.

(4) the antibacterial peptide is identified. The antibacterial peptide obtained above is analyzed by electrospray ionization mass spectrometry, the molecular weight shown in the mass spectrum (FIG. 1) is basically consistent with the theoretical molecular weight, and the purity of the antibacterial peptide is greater than 95%.

### Embodiment 3 Determination of the Antibacterial Activity of the Protease-Resistant Antibacterial Peptide VKLR

The minimum inhibitory concentration (micromole(s) per liter) of the antibacterial peptide VKLR is determined by the microdilution method. A diluent containing 0.01% acetic acid and 0.2% fetal bovine serum albumin is added to a 96-well plate, and a series of gradient antibacterial peptide solutions are prepared sequentially using the two-fold dilution method. Then, 100 microliters of each solution is placed in a 96-well cell culture plate, and then an equal volume of the bacterial solution to be tested (˜10⁵ colony forming units per milliliter) is added to each well. A positive control (containing bacterial solution but no antibacterial peptide) and a negative control (containing neither bacterial solution nor antibacterial peptide) are set up separately. The plate is incubated at a constant temperature of 37 degrees Celsius for 14-18 hours, and the optical absorbance value is measured at 492 nanometers (OD₄₉₂ₙₘ) using a microplate reader to determine the minimum inhibitory concentration. A measured value less than 0.1 is considered to indicate bacterial inhibition. Each test is performed in duplicate and repeated three times. The antibacterial activity of peptide VKLR against bacteria is detailed in Table 2.

TABLE 2

| | Minimum inhibitory |
|---|---|
| Strain | concentration (micromolar) |
| Escherichia coli 25922 | 2 |
| Escherichia coli K88 | 2 |
| Pseudomonas aeruginosa 15442 | 4 |
| Pseudomonas aeruginosa 27853 | 4 |
| Staphylococcus aureus 25923 | 2 |
| Staphylococcus aureus 6538 | 2 |
| Staphylococcus aureus 43300 | 4 |
| Staphylococcus epidermidis 49134 | 2 |

The results from Table 2 show that the antibacterial peptide VKLR has low minimum inhibitory concentrations against Escherichia coli, Pseudomonas aeruginosa, Staphylococcus aureus, and Staphylococcus epidermidis, demonstrating excellent broad-spectrum antibacterial activity.

### Embodiment 4 Determination of the Bactericidal Kinetics of the Antibacterial Peptide VKLR

Pseudomonas aeruginosa ATCC 15442 in the logarithmic growth phase is diluted to (1×10⁵) colony forming units per milliliter and incubated in phosphate-buffered saline (PBS) (10×10⁻³ molar, pH=7.4) with the antibacterial peptide VKLR at concentrations of 4×10⁻⁶ molar, 8×10⁻⁶ molar, and 16×10⁻⁶ molar. Similarly, Staphylococcus aureus ATCC 6538 cells in the logarithmic growth phase are diluted to (1×10⁶) colony forming units per milliliter and incubated in PBS (10×10⁻³ molar, pH=7.4) with the antibacterial peptide at concentrations of 2×10⁻⁶ molar, 4×10⁻⁶ molar, and 8×10⁻⁶ molar. Samples are taken at different time intervals (0, 3, 5, 15, 30, 60, and 120 minutes) and diluted to appropriate multiples. The diluted bacterial suspensions are plated on Mueller-Hinton Agar (MHA) plates. The plates are incubated in a 37 degrees Celsius incubator for 24 hours, after which the bacterial colonies are counted. Each test is performed in duplicate and independently repeated three times.

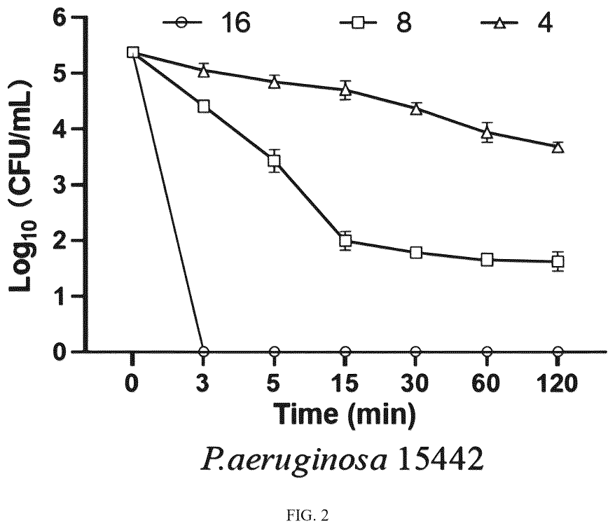

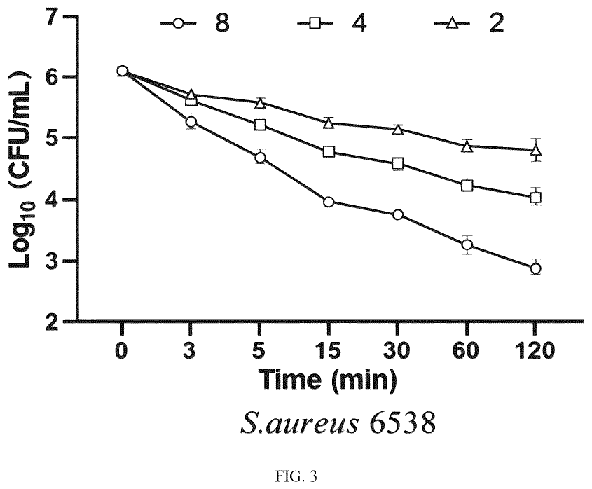

It may be seen from FIG. 2 that the antibacterial peptide VKLR may kill 99.9% of Pseudomonas aeruginosa within 15 minutes at bactericidal concentrations. It may be seen from FIG. 3 that the antibacterial peptide VKLR requires 120 minutes to kill 99.9% of Staphylococcus aureus at bactericidal concentrations. The reason is that Gram-positive bacteria have a dense peptidoglycan layer, which partly hinders the membrane-disrupting effect of the antibacterial peptide VKLR. In summary, the antibacterial effect of the antibacterial peptide VKLR on Gram-negative bacteria is stronger than that on Gram-positive bacteria.

### Embodiment 5 Analysis of the Effect of Antibacterial Peptide VKLR on the Outer Membrane Permeability of Pseudomonas aeruginosa

The fluorescent probe N-phenyl-1-naphthylamine (NPN) is used to measure the outer membrane permeability of Pseudomonas aeruginosa ATCC 15442 induced by the antibacterial peptide. Pseudomonas aeruginosa ATCC 15442 in the logarithmic phase is diluted with 5×10⁻³ molar 4-(2-hydroxyethyl)-1-piperazineethanesulfonic acid (HEPES) buffer until the optical density (OD) at 600 nanometers is 0.2. Then, the fluorescent dye NPN is added to the bacterial suspension of Pseudomonas aeruginosa at a final concentration of 10×10⁻⁶ molar. Next, 100 microliters of the bacterial suspension is mixed with 100 microliters of the peptide (final concentration range: 2×10⁻⁶ molar to 64×10⁻⁶ molar) in a 96-well plate. The fluorescence value is measured using a microplate reader at an excitation wavelength of 350 nanometers and an emission wavelength of 420 nanometers. The group treated with 10 micrograms per milliliter polymyxin B is used as the positive control, and the initial fluorescence of a bacterial suspension without peptide is used as the negative control. Each test is performed in duplicate and independently repeated three times.

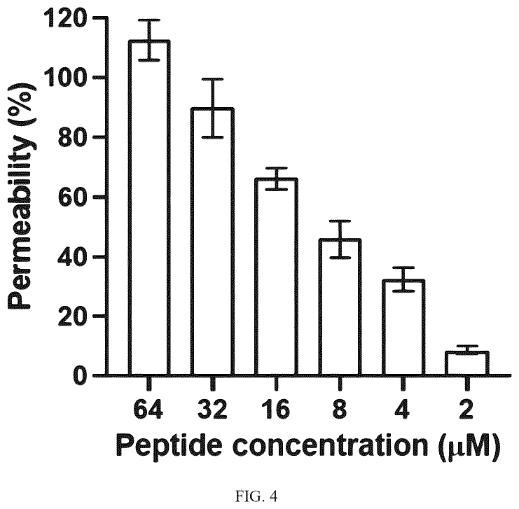

It may be seen from FIG. 4 that the effect of the antibacterial peptide VKLR on the outer membrane permeability of Pseudomonas aeruginosa is dose-dependent. At its minimum inhibitory concentration of 4×10⁻⁶ molar, it disrupts approximately 40% of the bacterial outer membrane.

### Embodiment 6 Test of the Binding Affinity of Antibacterial Peptide VKLR for Lipopolysaccharide (LPS)/Lipoteichoic Acid (LTA)

The fluorescent probe BODIPY-TR cadaverine (BC) is used to determine the ability of the antibacterial peptide VKLR to bind to the LPS of Pseudomonas aeruginosa and the LTA of Staphylococcus aureus. First, BC at a final concentration of 5×10⁻⁶ molar is incubated with LPS or LTA at a final concentration of 50×10⁻⁶ molar in the dark at 37 degrees Celsius for 4 hours. Next, the peptide VKLR solution is serially diluted in a 96-well plate to produce concentrations from 1×10⁻⁶ molar to 64×10⁻⁶ molar. Subsequently, the LPS or LTA probe is added to an equal volume of the peptide solution and incubated for 1 hour. Finally, the fluorescence intensity is measured using a microplate reader at an excitation wavelength of 580 nanometers and an emission wavelength of 620 nanometers. Each test is performed in duplicate and independently repeated three times.

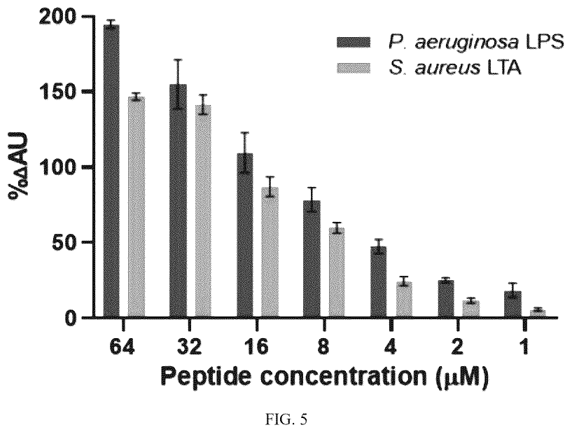

It may be seen from FIG. 5 that the binding of VKLR to the LPS of Pseudomonas aeruginosa and the LTA of Staphylococcus aureus is dose-dependent, and the binding to LPS is stronger. Based on the results of the binding kinetic analysis, VKLR shows a stronger binding capacity to LPS, which is one of the reasons for its faster bactericidal activity against Pseudomonas aeruginosa.

### Embodiment 7 the Effect of Antibacterial Peptide VKLR on the Degree of Bacterial Inner Membrane Depolarization

The lipophilic membrane probe 3,3′-dipropylthiadicarbocyanine iodide (DiSC₃-5) is used to determine the change in cytoplasmic membrane potential of Pseudomonas aeruginosa ATCC15442 and Staphylococcus aureus ATCC6538 induced by the peptide VKLR. Bacteria in the logarithmic growth phase are resuspended in 5×10⁻³ molar HEPES buffer (pH 7.4, containing 40×10⁻³ molar glucose and 200×10⁻³ molar KCl), and the OD is adjusted to 0.1 at a wavelength of 600 nanometers. Afterwards, 0.8×10⁻⁶ molar DiSC₃-5 is immediately added and incubated in the dark for 90 minutes. Meanwhile, the peptide VKLR solution is serially diluted in a 96-well plate to produce a concentration range from 8×10⁻⁶ molar to 16×10⁻⁶ molar. Next, the peptide solution is mixed with the bacterial suspension (1:1, volume by volume), and the fluorescence change at an excitation wavelength of 620 nanometers and an emission wavelength of 670 nanometers is immediately detected using a microplate reader.

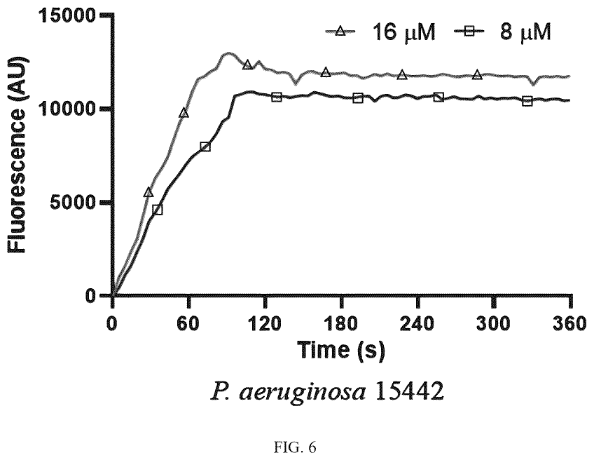

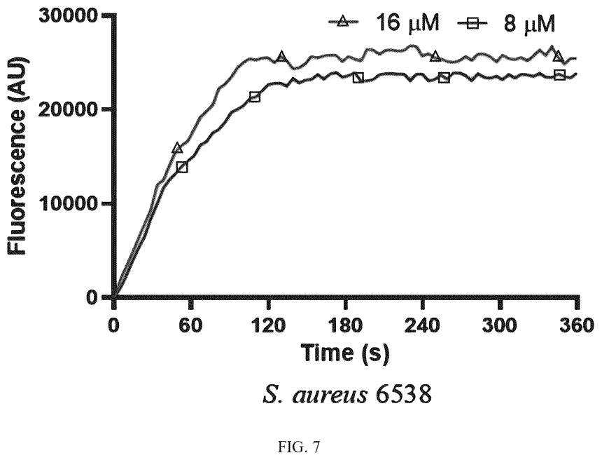

The fluorescent probe DiSC₃-5 is used as a tracer dye to evaluate changes in membrane potential. This environmentally sensitive cationic dye diffuses and accumulates in the phospholipid bilayer, leading to self-quenching of the dye. When VKLR alters the plasma membrane potential or structure, the fluorescence intensity is enhanced. It may be seen from FIG. 6 and FIG. 7 that the antibacterial peptide VKLR causes cytoplasmic membrane depolarization in a dose-dependent manner, and Pseudomonas aeruginosa reaches its peak faster under VKLR than Staphylococcus aureus, with its fluorescence peaking at 120 seconds.

### Embodiment 8 Determination of the Hemolytic Activity of the Antibacterial Peptide

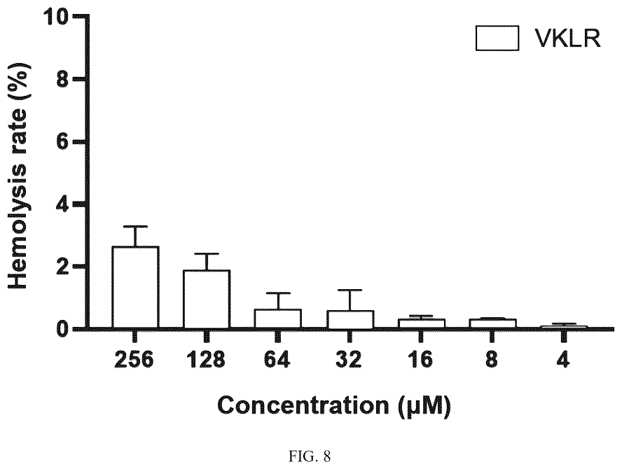

A 1% suspension of porcine red blood cells is purchased, centrifuged at 3000 revolutions per minute for 10 minutes, and the red blood cells are collected. The red blood cells are washed three times with PBS solution and then resuspended in 10 milliliters of PBS solution. 50 microliters of the red blood cell suspension is mixed thoroughly with 50 microliters of antibacterial peptide solutions at different concentrations, and the mixture is incubated at a constant temperature of 37 degrees Celsius for 1 hour. Subsequently, the mixture is centrifuged at 4 degrees Celsius and 3000 revolutions per minute for 10 minutes. The supernatant is taken, and the optical absorbance value is measured at 570 nanometers using a microplate reader. Here, 50 microliters of red blood cells mixed with 50 microliters of PBS solution is used as the negative control, and 50 microliters of red blood cells mixed with 50 microliters of 0.1% Tritonx-100 is used as the positive control. The minimum hemolytic concentration is defined as the concentration of the antibacterial peptide that causes 10% hemolysis, and the detection results are shown in FIG. 8. It may be seen from FIG. 8 that the antibacterial peptide VKLR does not exhibit hemolytic activity within the tested range. It causes 1% hemolysis of red blood cells at a concentration of 128 micromolar and fails to cause 10% hemolysis of red blood cells, indicating that the antibacterial peptide VKLR with low hemolytic activity has the potential to be developed as an antibiotic substitute.

In summary, the peptide VKLR, which is composed of natural amino acids, possesses excellent broad-spectrum antibacterial activity, strong salt stability, and protease stability, and has broad prospects for clinical application.

The embodiments described above are only for describing the optional modes of the present disclosure, and are not intended to limit the scope of the present disclosure. Without departing from the design spirit of the present disclosure, various modifications and improvements made to the technical schemes of the present disclosure by those of ordinary skill in the art shall fall within the protection scope defined by the claims of the present disclosure.

What is claimed is:

1. A short-chain antibacterial peptide VKLR, wherein the amino acid sequence of the short-chain antibacterial peptide VKLR is shown in SEQ ID NO. 1.

2. A broad-spectrum antibacterial agent, wherein the broad-spectrum antibacterial agent comprises the short-chain antibacterial peptide VKLR according to claim 1; and
an antibacterial activity is against at least one of Escherichia coli, Pseudomonas aeruginosa, Staphylococcus aureus, or Staphylococcus epidermidis.

3. An antibacterial composition, wherein the antibacterial composition comprises the short-chain antibacterial peptide VKLR according to claim 1; and
an antibacterial activity is against at least one of Escherichia coli, Pseudomonas aeruginosa, Staphylococcus aureus, or Staphylococcus epidermidis.


SEQUENCE LISTING (1 sequence)

SEQ ID NO: 1 - protein, 13 aa, synthetic construct
LVKLRVKLRVKLR